(19) United States
(12) Patent Application Publication
(10) Pub. No.: US 2025/0041316 A1
(43) Pub. Date: Feb. 6, 2025

(54) METHODS AND COMPOSITIONS FOR TREATING LIVER DISEASE

(71) Applicant: Temple University-Of The Commonwealth System of Higher Education, Philadelphia, PA (US)

(72) Inventor: Sheng Wu, Lafayette Hill, PA (US)

(21) Appl. No.: 18/793,238

(22) Filed: Aug. 2, 2024

Related U.S. Application Data
(60) Provisional application No. 63/517,690, filed on Aug. 4, 2023.

(51) Int. Cl. A61K 31/568; A61P 1/16; C12Q 1/6883; G01N 33/68

(52) U.S. Cl. CPC A61K 31/568; A61P 1/16; C12Q 1/6883; G01N 33/6893; C12Q 2600/118; C12Q 2600/158; G01N 2800/085; G01N 2800/50

(57) ABSTRACT

Women with polycystic ovary syndrome (PCOS) are predicted to have a higher risk for developing nonalcoholic fatty liver disease than normal women associated with insulin resistance and hypertrophy of adipose tissue. This invention provides improved compositions and methods for the treatment of liver disease in women including in women with PCOS.

### CROSS-REFERENCE TO RELATED APPLICATIONS

This application claims priority to U.S. Provisional Patent Application No. 63/517,690, filed Aug. 4, 2023, which is hereby incorporated herein by reference in its entirety.

### STATEMENT REGARDING FEDERALLY SPONSORED RESEARCH OR DEVELOPMENT

This invention was made with government support under R01 HD095512 awarded by the National Institutes of Health. The government has certain rights in the invention.

### BACKGROUND OF THE INVENTION

Hyperandrogenism is a major health issue affecting women of reproductive age, leading to reproductive and metabolic dysfunction. Metabolic dysfunction is associated with glucose intolerance and insulin resistance. Women with polycystic ovary syndrome (PCOS) are predicted to have a higher risk for developing nonalcoholic fatty liver disease than normal women associated with insulin resistance and hypertrophy of adipose tissue. However, elevated androgen alone does not cause fatty liver. There is a need in the art for improved compositions and methods for the treatment of liver disease in women, including women with PCOS. This invention satisfies this unmet need.

### SUMMARY OF THE INVENTION

In various aspects, the present invention provides a method of treating a liver disease or disorder, or a symptom thereof, in a subject in need of treatment, the method comprising administering to the subject at least one steroid hormone in an amount effective for treating the liver disease or disorder. In some embodiments, the subject is a woman, and the liver disease or disorder is non-alcoholic fatty liver disease (NAFLD), and currently named as metabolic associated fatty liver disease (MAFLD).

In some embodiments, the subject is a woman, and the woman further displays at least one clinical feature associated with polycystic ovary syndrome (PCOS). In some embodiments, the at least one clinical feature associated with PCOS comprises irregular or no menstrual periods, heavy periods, excess body and facial hair, acne, pelvic pain, difficulty getting pregnant, and patches of thick, darker, velvety skin, ovarian cysts, enlarged ovaries, excess androgen, weight gain, hirsutism, type 2 diabetes, obesity, obstructive sleep apnea, heart disease, mood disorders, endometrial cancer, hyperandrogenism, or any combination thereof. In some embodiments, the liver disease or disorder comprises non-alcoholic fatty liver disease (NAFLD), nonalcoholic steatohepatitis (NASH), liver fibrosis, alcoholic liver disease, hepatitis, NASH with cirrhosis, and comorbidities of testosterone abnormalities. In some embodiments, the liver disease or disorder comprises NAFLD. In some embodiments, the at least one steroid hormone comprises dihydrotestosterone (DHT). In some embodiments, the method restores the level of at least one biomarker of the liver disease or disorder in the subject. In some embodiments, the at least one biomarker comprises hepatic protein or mRNA levels of Cluster of Differentiation 36 (CD36), Very Low Density Lipoprotein Receptor (VLDLR), Carboxylesterase 3 (Ces3), Regucalcin (RGN), Prolow-density lipoprotein receptor-related protein 1 (LRP1), and hepatic levels triglyceride (TG) and cholesterol levels.

In various aspects, the present invention provides a composition for treating a liver disease or disorder in a subject in need thereof, wherein the composition comprises at least one steroid hormone. In some embodiments, the subject is a woman, and the liver disease or disorder is non-alcoholic fatty liver disease (NAFLD). In some embodiments, the subject is a woman, and the woman further displays at least one clinical feature associated with PCOS. In some embodiments, the at least one clinical feature associated with PCOS comprises irregular or no menstrual periods, heavy periods, excess body and facial hair, acne, pelvic pain, difficulty getting pregnant, and patches of thick, darker, velvety skin, ovarian cysts, enlarged ovaries, excess androgen, weight gain, hirsutism, type 2 diabetes, obesity, obstructive sleep apnea, heart disease, mood disorders, endometrial cancer, hyperandrogenism, or any combination thereof. In some embodiments, the liver disease or disorder comprises NAFLD, NASH, liver fibrosis, alcoholic liver disease, hepatitis, NASH with cirrhosis, and comorbidities of testosterone abnormalities. In some embodiments, the liver disease or disorder comprises NAFLD. In some embodiments, the at least one steroid hormone comprises DHT. In some embodiments, the composition restores the level of at least one biomarker of the liver disease or disorder in the subject following administration to the subject. In some embodiments, the e at least one biomarker comprises hepatic protein or mRNA levels of CD36, VLDLR, Ces3, RGN, and LRP1, and hepatic TG and cholesterol levels. In some embodiments, the composition is a pharmaceutical composition. In some embodiments, the composition further comprises at least one pharmacologically acceptable excipient.

In various aspects, the present invention provides a method of preventing a subject at risk of developing a liver disease or disorder from developing the liver disease or disorder, the method comprising: a) identifying a subject at risk, wherein the identifying comprises: i) measuring the level of at least one biomarker associated with the liver disease or disorder in a subject; ii) comparing the level of the at least one biomarker to determine to normal levels of the at least one biomarker; and iii) determining that the subject is at risk if the levels of the at least one biomarker is above or below the normal levels; and b) administering to the subject at risk at least one steroid hormone in an amount effective for treating the liver disease or disorder. In some embodiments, the subject is a woman, and the liver disease or disorder is non-alcoholic fatty liver disease (NAFLD). In some embodiments, the subject is a woman, and the woman further displays at least one clinical feature associated with PCOS. In some embodiments, the at least one clinical feature associated with PCOS comprises irregular or no menstrual periods, heavy periods, excess body and facial hair, acne, pelvic pain, difficulty getting pregnant, and patches of thick, darker, velvety skin, ovarian cysts, enlarged ovaries, excess androgen, weight gain, hirsutism, type 2 diabetes, obesity, obstructive sleep apnea, heart disease, mood disorders, endometrial cancer, hyperandrogenism, or any combination thereof. In some embodiments, the liver disease or disorder comprises NAFLD. In some embodiments, the at least one steroid hormone comprises DHT. In some embodiments, the method of preventing a liver disease or disorder comprises restoring levels of the at least one biomarker in the subject at risk. In some embodiments, the at least one biomarker comprises hepatic protein or mRNA levels of CD36, VLDLR, Ces3, RGN, and LRP1, and hepatic TG and cholesterol levels.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1C, presents representative data demonstrating that treatment with dihydrotestosterone (DHT) reduced fatty liver content in female mice fed with a Western diet. FIG. 1A depicts a representative illustration of the experimental system used to study the effects of DHT on metabolism. Mice were fed with a Western diet (WD). At 45 days of age, one group of mice were administered DHT via pellet at Week 0 and at Week 4-5 (WD+DHT). One group of WD fed mice were not administered with DHT (WD). Mice were euthanized at Week 8. Ex vivo lipolysis, histology and molecular studies were performed. FIG. 1B presents representative data depicting body weight measurement of mice (WD and WD+DHT) demonstrating that WD mice were lighter than WD+DHT mice. FIG. 1C depicts representative images of the livers of WD and WD+DHT mice demonstrating lower levels of fat in WD+DHT mice compared to WD mice.

FIG. 2, comprising FIG. 2A and FIG. 2B, presents representative data demonstrating lower levels of triglyceride (FIG. 2A) and cholesterol (FIG. 2B) in WD+DHT mice compared to WD mice.

FIG. 3, comprising FIG. 3A through FIG. 3C, presents representative data depicting demonstrating lower levels of CD36 (FIG. 3A), VLDR (FIG. 3B), and Ces3b (FIG. 3C) gene expression in WD+DHT mice compared to WD mice, as assayed by qRT-PCR.

FIG. 4 presents representative data demonstrating decreased lipid deposition in WD+DHT mice compared to WD mice as assessed by oil red (top panel) and H&E staining (lower panel).

FIG. 5 presents representative data demonstrating that liver shows lower triglyceride after mice are fed with WD for 8 weeks and then treated with DHT for 3 weeks under WD condition (11 weeks total WD), compared to liver of WD alone for 11 weeks.

### DETAILED DESCRIPTION

The present invention is based, in part, on the unexpected findings that administering dihydrotestosterone (DHT) to female mice fed with obesity-inducing western diet (WD) resulted in reduced levels of liver-associated triglycerides (TGs) and cholesterol, and significantly altered levels of genes related to fatty acid transport into the liver and TG secretion from the liver (e.g., Cluster of Differentiation 36 (CD36) and regucalcin (RGN)) demonstrating that the liver of mice administered with TG adopts a protective mechanism with dramatically decreased lipid storage. Accordingly, the present invention provides compositions and methods for the treatment of liver disease in females, including females with polycystic ovary syndrome (PCOS).

In one embodiment, the present invention comprises a method of treating a liver disease or disorder, or a symptom thereof, in a subject in need of treatment, the method comprising administering to the subject at least one steroid hormone in an amount effective for treating the liver disease or disorder. In one embodiment the liver disease or disorder comprises non-alcoholic fatty liver disease (NAFLD). In one embodiment, the subject is a woman. In one embodiment, the subject is a woman with NAFLD. In one embodiment, the subject is a woman who displays at least one sign or symptom associated with PCOS. In one embodiment, the at least one sign or symptom associated with PCOS comprises hyperandrogenism. In one embodiment, the at least one steroid hormone comprises DHT. In one embodiment, treating the liver disease or disorder, or a symptom thereof, in a subject in need of treatment, comprises restoring the level of at least one biomarker of the liver disease or disorder to normal level, wherein the at least one biomarker comprises protein and/or mRNA levels of Cluster of Differentiation 36 (CD36), Very Low Density Lipoprotein Receptor (VLDLR), Carboxylesterase 3 (Ces3), Regucalcin (RGN), Prolow-density lipoprotein receptor-related protein 1 (LRP1), and/or levels of hepatic triglyceride (TG) and hepatic cholesterol.

In one embodiment, the present invention comprises compositions for treating a liver disease or disorder in a subject in need thereof, wherein compositions comprise at least one steroid hormone. In one embodiment, the at least one steroid hormone comprises DHT. In one embodiment the liver disease or disorder comprises non-alcoholic fatty liver disease (NAFLD). In one embodiment, the subject is a woman. In one embodiment, the subject is a woman with NAFLD. In one embodiment, the subject is a woman who displays at least one sign or symptom associated with PCOS. In one embodiment, the at least one sign or symptom associated with PCOS comprises hyperandrogenism. In one embodiment, treating a liver disease or disorder in a subject in need thereof, comprises restoring the level of at least one biomarker of the liver disease or disorder to normal level, wherein the at least one biomarker comprises hepatic protein or mRNA levels of CD36, VLDLR, Ces3, RGN, and LRP1, and hepatic TG and cholesterol levels.

In one embodiment, the present invention comprises a method of treating a liver disease or disorder, or a symptom thereof, in a subject in need of treatment, the method comprising administering to the subject at least one composition of the present invention and thereby restoring the level of at least one biomarker of the liver disease or disorder to normal level, wherein the at least one biomarker comprises hepatic protein or mRNA levels of CD36, VLDLR, Ces3, RGN, and LRP1, and hepatic TG and cholesterol levels. In one embodiment, the at least one steroid hormone comprises DHT. In one embodiment the liver disease or disorder comprises non-alcoholic fatty liver disease (NAFLD). In one embodiment, the subject is a woman. In one embodiment, the subject is a woman with NAFLD. In one embodiment, the subject is a woman who displays at least one sign or symptom associated with PCOS. In one embodiment, the at least one sign or symptom associated with PCOS comprises hyperandrogenism.

In one embodiment, the present invention comprises a method of treating a subject at risk of developing a liver disease or disorder, or a symptom thereof, the method comprising identifying a subject at risk of developing a liver disease or disorder by measuring the level of at least one biomarker associated with the liver disease or disorder, comparing the level of the at least one biomarker to determine if it is above or below a normal level of the at least one biomarker, and administering to the subject a pharmaceutical composition comprising at least one steroid hormone in amount effective to restore the levels of the at least one biomarker to normal level. In one embodiment, the at least one steroid hormone comprises DHT. In an embodiment, the biomarker comprises hepatic protein or mRNA levels of CD36, VLDLR, Ces3, RGN, and LRP1, and hepatic TG and cholesterol levels. In one embodiment the liver disease or disorder comprises non-alcoholic fatty liver disease (NAFLD). In one embodiment, the subject at risk is a woman. In one embodiment, the subject at risk is a woman with NAFLD. In one embodiment, the subject at risk is a woman who displays at least one sign or symptom associated with PCOS. In one embodiment, the at least one sign or symptom associated with PCOS comprises hyperandrogenism. In one aspect, the at least one biomarker further comprises ALP, ALT, AST, GGT, LDL, liver biopsy, inflammation biomarkers, non-HDL cholesterol, hematocrit, hemoglobin, lipoprotein phospholipase A2, bilirubin, albumin, SHBG, imaging biomarkers, liver histology biomarkers, biomarkers in the literature related to the disease and condition described herein, and liver damage biomarkers.

In one aspect of this embodiment, the subject has one or more of the following comorbidities: obesity, type 2 diabetes, dyslipidemia, cardiovascular disease, thyroid dysfunction, chronic kidney disease, liver disease, osteoporosis, hypogonadism, hypertension, sarcopenia, or cachexia.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A disease or disorder is “alleviated” if the severity of a sign or symptom of the disease or disorder, the frequency with which such a sign or symptom is experienced by a patient, or both, is reduced. “Alleviating” specific cancers and/or their pathology includes degrading a tumor, for example, breaking down the structural integrity or connective tissue of a tumor, such that the tumor size is reduced when compared to the tumor size before treatment. “Alleviating” metastasis of cancer includes reducing the rate at which the cancer spreads to other organs.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Parenteral” administration of an immunogenic composition includes, e.g., subcutaneous (s.c.), intravenous (i.v.), intramuscular (i.m.), or intrasternal injection, or infusion techniques.

“Sample” or “biological sample” as used herein means a biological material from a subject, including but is not limited to organ, tissue, exosome, blood, plasma, saliva, urine and other body fluid. A sample can be any source of material obtained from a subject.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

The term “therapeutic” as used herein means a treatment and/or prophylaxis. A therapeutic effect is obtained by suppression, remission, or eradication of a disease state.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

The terms “serum testosterone” or “serum (17-β)-Hydroxy-4-Androsten-3-one levels,” “serum T levels,” “serum testosterone concentration,” “plasma testosterone concentration,” “testosterone concentration in the blood,” and “serum testosterone concentration,” are used interchangeably and refer to the “total” testosterone concentration which is the sum of the bioavailable testosterone including free and bound testosterone concentrations. Unless otherwise specified, these values are “observed” testosterone concentrations without adjusting or correcting for the base-line serum testosterone levels in the subject(s). As with any bio-analytical measure, for increased consistency, the method employed to measure initial serum testosterone levels should be consistent with the method used to monitor and re-measure serum testosterone levels during clinical testing and testosterone therapy for a subject. Unless otherwise stated, “testosterone concentration” refers to serum total testosterone concentration.

Average serum testosterone concentrations can be determined using methods and practices known in the art. For example, the average baseline plasma testosterone concentration of a human male is the arithmetic mean of the total plasma testosterone concentration determined on at least two consecutive time points that are reasonably spaced from each other, for example from about 1 hour to about 168 hours apart. In a particular case, the plasma testosterone concentration can be determined on at least two consecutive times that are about 12 hours to about 48 hours apart. In another particular method, the plasma testosterone concentration of the human male can be determined at a time between about 5 o'clock and about 11 o'clock in the morning. Further, the plasma testosterone concentration can be the determined by standard analytical procedures and methods available in the art, such as for example, automated or manual immunoassay methods, liquid chromatography or liquid chromatography-tandem mass spectrometry (LC-MSMS) etc.

As used herein, “free testosterone serum concentration”, refers to the testosterone concentration not bound to protein e.g., SHBG or albumin. In some aspects of the methods described herein, free testosterone serum concentrations are used instead of serum total testosterone concentrations. For example, a subject can appear to have total serum testosterone levels in the normal range but can be still considered hypogonadal or testosterone deficient based on free testosterone levels.

As used herein, “in need of treatment” refers to a subject that has a disease or is suspected of having the disease according to various diagnostic criteria typically used in practice, or desires treatment or is indicated for treatment. Thus, “in need of treatment” can include the step of identifying a subject in need of treatment.

As used herein, “identifying a subject in need of treatment” can include the step of obtaining a biological sample from the subject and determining the level of one or more biomarkers as described herein, assessing the histology of a biological sample obtained from said subject, performing an imaging analysis on the subject, assessing one or more clinical characteristics of said subject (e.g., assessing symptoms or overt symptoms), or a combination thereof.

As used herein, the term AUCt1-t2 is the area under the curve of a plasma-versus-time graph determined for the analyte from the time “t1 to time t2”. Wherein t1 and t2 are times (in hours) post dosing. For Example, t1 could be 1 hour and t2 could be 2 hours.

The terms “biomarker” and “marker” are used herein interchangeably. They refer to a substance that is a distinctive indicator of a biological process, biological event and/or pathologic condition. A biomarker may refer to a peptide, polypeptide, protein, nucleic acid, lipid, steroid, carbohydrate, or any known biological molecule.

The phrase “biological sample” is used herein in its broadest sense. A sample may be of any biological tissue or fluid from which biomarkers of the present invention may be assayed. Examples of such samples include but are not limited to blood, lymph, urine, gynecological fluids, biopsies, amniotic fluid and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, and archival samples with known diagnosis, treatment and/or outcome history. Biological or body samples may also include sections of tissues such as frozen sections taken for histological purposes. The sample also encompasses any material derived by processing a biological or body sample. Derived materials include, but are not limited to, cells (or their progeny) isolated from the sample, proteins or nucleic acid molecules extracted from the sample. Processing of a biological or body sample may involve one or more of: filtration, distillation, extraction, concentration, inactivation of interfering components, addition of reagents, and the like.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold higher or more, as compared with a control.

“Differentially decreased expression” or “down regulation” refers to biomarker product levels which are at least 10% or more, for example, 20%, 30%, 40%, or 50%, 60%, 70%, 80%, 90% lower or less, and/or 0.9 fold, 0.8 fold, 0.6 fold, 0.4 fold, 0.2 fold, 0.1 fold or less, as compared with a control.

As used herein, the term “Cavg,” “Cave,” or “C-average” are used interchangeably, and is determined as the AUCt1-t2 mean AUC divided by the time period (|t1-t2|). For example, Cavg t0-t8 is the average plasma concentration over a period of 8 hours from t1=0 to t2=8 hours) post-dosing determined by dividing the AUCt0-t8 value by 8. Similarly, Cavg t0-t12 is the average plasma concentration over a period of 12 hours post-dosing determined by dividing the AUCt0-t12 value by 12 (t1=0-t2=12). Similarly, Cavg t12-t24 is the average plasma concentration over a period of 12 hours post-dosing determined by dividing the AUCt12-t24 value by 12 (t1=12-t2=24); Cavg-t24 is the average plasma concentration over a period of 24 hours post-dosing determined by dividing the AUCt0-t24 value by 24 (t1=0-t2=24), and so on. Unless otherwise stated, all Cavg values are considered to be Cavg-t24 and unless otherwise stated, all the time values are expressed in hours (h). For example, the term Cavg t0-t24 denotes Cavg from time zero (0) to 24 hours post dosing.

“Androgen receptor agonists” as used herein refers to compounds or molecules such as testosterone that bind and activate the androgen receptor. Androgen receptor agonists include, but are not limited to, dihydrotestosterone, mibolerone, testosterone, methyltrienolone, oxandrolone, nandrolone and fluoxymesterone. Where appropriate, fatty acid esters of these androgen receptor agonists can be used herein accordingly.

“Testosterone ester” as used herein refers to testosterone esterified with a fatty acid and includes but is not limited to, testosterone undecanoate, testosterone tridecanoate, testosterone enanthate, testosterone decanoate, testosterone palmitate, testosterone cypionate, and testosterone propionate. In one aspect, a preferred testosterone ester for oral administration according to the methods and compositions described herein is testosterone undecanoate. In one aspect, a preferred testosterone ester for oral administration according to the methods and compositions described herein is testosterone decanoate. In one aspect, a preferred testosterone ester for oral administration according to the methods and compositions described herein is testosterone dodecanoate. In one aspect, a preferred testosterone ester for oral administration according to the methods and compositions described herein is testosterone tridecanoate. In one aspect, a preferred testosterone ester for oral administration according to the methods and compositions described herein is testosterone tetradecanoate.

“Fibrosis,” or “Liver Fibrosis” as used herein, refers to the accumulation of extracellular matrix constituents that occurs following trauma, inflammation, tissue repair, immunological reactions, cellular hyperplasia, and neoplasia.

“Normal Range” as used herein refers to a range of values generally considered to be representative of a healthy individual or population. It is understood that “normal range” is general specific to a type of assay or lab. For example, for a particular analyte, a normal range for one assay or lab can differ as compared to a normal range for another assay or lab for the same analyte. Thus, the normal ranges described herein may vary from lab to lab or assay to assay. The skilled artisan understands that the normal ranges disclosed herein can vary from individual to individual. The skilled artisan understands that the normal ranges described herein are typically appropriate for the indicated sample e.g., serum and that other samples e.g., saliva can have different normal ranges.

“Upper Normal Range” as used herein refers to above the 50% level for that range. For example, a biomarker may have a normal range of 10-40 U/L, according to this definition, the upper normal range is above 25 U/L and below 40 U/L. In another example, consider a biomarker having a range of 43-115 U/L, the high normal range is above 79 U/L and below 115 U/L.

As used herein, “ALT normal range” refers to the range of serum alanine transaminase values considered normal for healthy individuals and is from about 10-40 U/L according to the assay/lab used in Example 1.

As used herein, “AST normal range” refers to the range of serum aspartate transaminase values considered normal for healthy individuals and is from about 10-43 U/L according to the assay/lab used in Example 1.

As used herein, “ALP normal range” refers to the range of serum alkaline phosphatase values considered normal for healthy individuals and is from about 43-115 U/L according to the assay/lab used in Example 1.

As used herein, “GGT normal range” refers to the range of serum gamma-glutamyl transferase values considered normal for healthy individuals and is from about 10-49 U/L according to the assay/lab used in Example 1.

As used herein, “triglyceride normal range” refers to the range of serum triglyceride values considered normal for healthy individuals and is from about 45-200 mg/dL according to the assay/lab used in Example 1.

As used herein, “LDL normal range” and is from about 50-160 mg/dL according to the assay/lab used in Example 1.

As used herein, “desirable total cholesterol” or “total cholesterol normal range” and for adults is from about 125-200 mg/dL.

As used herein, “non-HDL cholesterol normal range” for adults is 130-159 mg/dL (3.4-4.0 mmol/L) is considered near ideal. The skilled artisan realizes that an ideal level depends on a number of factors and the normal range is variable depending on these factors.

As used herein, “VLDL” refers to very low density lipoprotein and has a normal range of 2 to 30 mg/dL.

As used herein, “SHBG” refers to sex hormone binding globulin and has a normal range of about 20 to 60 nmol/L for healthy adult males. As with any other of the biomarkers disclosed herein the normal ranges depend on a number of factors including sex and age e.g., Adult female, premenopausal 40-120 nmol/L; Adult female, postmenopausal 28-112 nmol/L; Adult male; 20-60 nmol/L; Infant (1-23 months) 60-252 nmol/L; Prepubertal (2 years-8 years) 72-220 nmol/L; Pubertal female 36-125 nmol/L; and Pubertal male 16-100 nmol/L.

As used herein, terms such as fatty liver disease, liver fibrosis, alcoholic liver disease, hepatitis, NAFLD, NASH, and NASH with cirrhosis are given their customary meaning to one of ordinary skill in the art and such condition are identifiable and diagnosable by medical professionals such as physicians, hepatologists, gastroenterologists and the like based on assessment of the relevant disease characteristics in patients.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one embodiment, the present invention comprises a method of treating a liver disease or disorder, or a symptom thereof, in a subject in need of treatment, the method comprising administering to the subject at least one steroid hormone in an amount effective for treating the liver disease or disorder. In one embodiment the liver disease or disorder comprises non-alcoholic fatty liver disease (NAFLD). In one embodiment, the subject is a woman. In one embodiment, the subject is a woman with NAFLD. In one embodiment, the subject is a woman who displays at least one sign or symptom associated with PCOS. In one embodiment, the at least one sign or symptom associated with PCOS comprises hyperandrogenism. In one embodiment, the at least one steroid hormone comprises DHT. In one embodiment, treating the liver disease or disorder, or a symptom thereof, in a subject in need of treatment, comprises restoring the level of at least one biomarker of the liver disease or disorder to normal level, wherein the at least one biomarker comprises protein and/or mRNA levels of Cluster of Differentiation 36 (CD36), Very Low Density Lipoprotein Receptor (VLDLR), Carboxylesterase 3 (Ces3), Regucalcin (RGN), Prolow-density lipoprotein receptor-related protein 1 (LRP1), and/or levels of hepatic triglyceride (TG) and hepatic cholesterol.

In one embodiment, the present invention comprise compositions for treating a liver disease or disorder in a subject in need thereof, wherein the compositions comprise at least one steroid hormone. In one embodiment, the at least one steroid hormone comprises DHT. In one embodiment the liver disease or disorder comprises non-alcoholic fatty liver disease (NAFLD). In one embodiment, the subject is a woman. In one embodiment, the subject is a woman with NAFLD. In one embodiment, the subject is a woman who displays at least one sign or symptom associated with PCOS. In one embodiment, the at least one sign or symptom associated with PCOS comprises hyperandrogenism. In one embodiment, treating a liver disease or disorder in a subject in need thereof, comprises restoring the level of at least one biomarker of the liver disease or disorder to normal level, wherein the at least one biomarker comprises hepatic protein or mRNA levels of CD36, VLDLR, Ces3, RGN, and LRP1, and hepatic TG and cholesterol levels.

In one embodiment, the present invention comprises a method of treating a liver disease or disorder, or a symptom thereof, in a subject in need of treatment, the method comprising administering to the subject at least one composition of the present invention and thereby restoring the level of at least one biomarker of the liver disease or disorder to normal level, wherein the at least one biomarker comprises hepatic protein or mRNA levels of CD36, VLDLR, Ces3, RGN, and LRP1, and hepatic TG and cholesterol levels. In one embodiment, the at least one steroid hormone comprises DHT. In one embodiment the liver disease or disorder comprises non-alcoholic fatty liver disease (NAFLD). In one embodiment, the subject is a woman. In one embodiment, the subject is a woman with NAFLD. In one embodiment, the subject is a woman who displays at least one sign or symptom associated with PCOS. In one embodiment, the at least one sign or symptom associated with PCOS comprises hyperandrogenism.

In one embodiment, the present invention comprises a method of treating a subject at risk of developing a liver disease or disorder, or a symptom thereof, the method comprising identifying a subject at risk of developing a liver disease or disorder by measuring the level of at least one biomarker associated with the liver disease or disorder, comparing the level of the at least one biomarker to determine if it is above or below a normal level of the at least one biomarker, and administering to the subject a pharmaceutical composition comprising at least one steroid hormone in amount effective to restore the levels of the at least one biomarker to normal level. In one embodiment, the at least one steroid hormone comprises DHT. In an embodiment, the biomarker comprises hepatic protein or mRNA levels of CD36, VLDLR, Ces3, RGN, and LRP1, and hepatic TG and cholesterol levels. In one embodiment the liver disease or disorder comprises non-alcoholic fatty liver disease (NAFLD). In one embodiment, the subject at risk is a woman. In one embodiment, the subject at risk is a woman with NAFLD. In one embodiment, the subject at risk is a woman who displays at least one sign or symptom associated with PCOS. In one embodiment, the at least one sign or symptom associated with PCOS comprises hyperandrogenism.

In one aspect, the at least one biomarker further comprises ALP, ALT, AST, GGT, LDL, liver biopsy, inflammation biomarkers, non-HDL cholesterol, hematocrit, hemoglobin, lipoprotein phospholipase A2, bilirubin, albumin, SHBG, imaging biomarkers, liver histology biomarkers, biomarkers in the literature related to the disease and condition described herein, and liver damage biomarkers.

In one aspect of this embodiment, the subject has one or more of the following comorbidities: obesity, type 2 diabetes, dyslipidemia, cardiovascular disease, thyroid dysfunction, chronic kidney disease, liver disease, osteoporosis, hypogonadism, hypertension, sarcopenia, or cachexia.

### Liver Disease or Disorder

In one embodiment, the present invention comprises methods and compositions for treating a liver disease or disorder, or a symptom thereof, in a subject in need of treatment, the method comprising administering to the subject at least one steroid hormone in an amount effective for treating the liver disease or disorder. In one embodiment, the liver disease or disorder comprises a fatty liver disease. In one embodiment, the liver disease or disorder comprises nonalcoholic fatty liver disease (NAFLD). In one embodiment, the subject is a woman. In one embodiment, the subject is a woman with NAFLD.

Nonalcoholic fatty liver disease (NAFLD) may also result from metabolic disorders such as, e.g., galactosemia, glycogen storage diseases, homocystinuria, and tyrosemia, as well as dietary conditions such as malnutrition, total parenteral nutrition, starvation, and overnutrition. In certain cases, NAFLD is associated with jejunal bypass surgery. Other causes include exposure to certain chemicals such as, e.g., hydrocarbon solvents, and certain medications, such as, e.g., amiodarone, corticosteroids, estrogens (e.g., synthetic estrogens), tamoxifen, maleate, methotrexate, nucleoside analogs, and perhexiline. Acute fatty liver conditions can also arise during pregnancy.

In some embodiments, the fatty liver disease is characterized by the baseline pretreatment level in the subject of at least one criteria selected from the group consisting of ALT in a range of 10 to 300 IU/L, AST in a range of 10 to 250 IU/L, HDL/C in a range of 25 to 55 mg/dl, LDL-C in a range of 100 to 200 mg/dl, triglycerides in a range of 100 to 1000 mg/dl, TC in a range of 170 to 300 mg/dl, High TG and low HDL-C, TG/HDL-C ratio in a range of 3.75 to 10, non-HDL-C in a range of 100 to 250 mg/dl, Free fatty acid in a range of 400 to 1000μ Eq/L, HOMA-IR in a range of 1.5 to 5, HbA1c in a range of 5.7 to 10%, Fasting plasma glucose in a range of 100 to 200 mg/dl, impaired glucose tolerance and metabolic syndrome. The fatty liver disease can be characterized by the baseline pretreatment level in the subject of at least one criteria selected from the group consisting of low level of EPA, docosapentaenoic acid (DPA), docosahexaenoic acid (DHA), EPA/arachidonic acid (AA), DHA/AA. DHA/DPA, AA/Homo-γ-linoleic acid: and high level of AA, monounsaturated fatty acid (MUFA), palmitoleic acid, oleic acid, oleic acid/stearic acid, palmitoleic acid/palmitic acid, γ-linoleic acid/linoleic acid, adrenic acid/AA compared to each average level in subjects with fatty liver disease.

In some embodiments, the fatty liver disease is selected from the group comprising NAFLD, non-Alcoholic Steatohepatitis (NASH), liver fibrosis, alcoholic liver disease, hepatitis, NASH with cirrhosis, and comorbidities of testosterone abnormalities.

It was surprisingly discovered that oral DHT (or androgen) therapy e.g., oral administration of a pharmaceutical composition containing a DHT is particularly useful for treating liver disease. For example, it was found that the instant methods and compositions reduce relevant biomarker levels related to liver disease (e.g., fatty liver disease, liver fibrosis, alcoholic liver disease, hepatitis, NAFLD, NASH, NASH with cirrhosis, and comorbidities of testosterone deficiency). Unexpectedly, the methods and compositions disclosed herein, substantial reductions (or improvements) in triglyceride levels, cholesterol levels, biomarkers of liver injury, and biomarkers for other diseases and conditions, a biomarker of cardiovascular disease.

Without wishing to be bound by theory, it is believed that in some contexts, as illustrated herein, subjects with PCOS with specific biomarkers in the high normal range or above normal are particularly sensitive to the methods and compositions described herein and see surprising reductions or improvements in the levels of these biomarkers. Again, in many cases these biomarker improvements are both over baseline values and in comparison to an active control. Moreover, populations of individuals that are at higher risk for developing specific diseases and conditions are particularly amenable to the treatments described herein. Thus, the methods and compositions described herein improve or ameliorate biomarkers associated with diseases by 5%, 10%, 15%, 20%, 25%, 30%, 40%, 50%, 60%, or 70% or more compared to baseline values. It is also believed that the method and compositions described herein have utility and can be used in subjects that do not have PCOS.

It was unexpectedly found that normalization or improvement of biomarkers (e.g., one or more biomarkers) can occur relatively quickly after commencement of the methods using the compositions described herein. For example, treatment for 1, 2, 3, 4, 5, 6, or 7 or more weeks can provide substantial improvements in one or more biomarkers associated with liver disease or mortality. Moreover, the methods and compositions provide sustained responses e.g., 1, 2, 3, 4, 5, 6, 7, 8, 9, 10, 11 or 12 months or more. As illustrated in the Examples, treatment with the methods and compositions described herein can result in substantial improvements of biomarker levels.

### Polycystic Ovary Syndrome (PCOS)

In one embodiment, the present invention comprises methods and compositions for treating a liver disease or disorder, or a symptom thereof, in a subject in need of treatment, the method comprising administering to the subject at least one steroid hormone in an amount effective for treating the liver disease or disorder. In one embodiment, the subject is a woman who displays at least one sign or symptom associated with PCOS.

In one embodiment, the subject additionally displays at least one sign or symptom associated with PCOS. In one embodiment the at least one sign or symptom associated with PCOS comprises any sign or symptom associated with hyperandrogenism such as, but not limited to, hirsutism, alopecia, masculine appearance, hidradenitis suppurativa, oligomenorrhea, acne, obesity, infertility, deepening of voice, oily skin, seborrhea, libido, type 2 diabetes, or any combination thereof.

In one embodiment the at least one sign or symptom associated with PCOS comprises irregular or no menstrual periods, heavy periods, excess body and facial hair, acne, pelvic pain, difficulty getting pregnant, and patches of thick, darker, velvety skin, ovarian cysts, enlarged ovaries, excess androgen, weight gain, hirsutism, type 2 diabetes, obesity, obstructive sleep apnea, heart disease, mood disorders, endometrial cancer, hyperandrogenism, or any combination thereof.

### Methods

In one embodiment, the present invention comprises a method of treating a liver disease or disorder, or a symptom thereof, in a subject in need of treatment, the method comprising administering to the subject at least one steroid hormone in an amount effective for treating the liver disease or disorder. In one embodiment the liver disease or disorder comprises non-alcoholic fatty liver disease (NAFLD). In one embodiment, the subject is a woman. In one embodiment, the subject is a woman with NAFLD. In one embodiment, the subject is a woman who displays at least one sign or symptom associated with PCOS. In one embodiment, the at least one sign or symptom associated with PCOS comprises hyperandrogenism. In one embodiment, the at least one steroid hormone comprises DHT. In one embodiment, treating the liver disease or disorder, or a symptom thereof, in a subject in need of treatment, comprises restoring the level of at least one biomarker of the liver disease or disorder to normal level, wherein the at least one biomarker comprises hepatic protein or mRNA levels of CD36, VLDLR, Ces3, RGN, and LRP1, and hepatic TG and cholesterol levels.

In one embodiment, the present invention comprises a method of treating a liver disease or disorder, or a symptom thereof, in a subject in need of treatment, the method comprising administering to the subject at least one composition of the present invention and thereby restoring the level of at least one biomarker of the liver disease or disorder to normal level, wherein the at least one biomarker comprises hepatic protein or mRNA levels of CD36, VLDLR, Ces3, RGN, and LRP1, and hepatic TG and cholesterol levels. In one embodiment, the at least one steroid hormone comprises DHT. In one embodiment the liver disease or disorder comprises non-alcoholic fatty liver disease (NAFLD). In one embodiment, the subject is a woman. In one embodiment, the subject is a woman with NAFLD. In one embodiment, the subject is a woman who displays at least one sign or symptom associated with PCOS. In one embodiment, the at least one sign or symptom associated with PCOS comprises hyperandrogenism.

In one embodiment, the present invention comprises a method of treating a subject at risk of developing a liver disease or disorder, or a symptom thereof, the method comprising identifying a subject at risk of developing a liver disease or disorder by measuring the level of at least one biomarker associated with the liver disease or disorder, comparing the level of the at least one biomarker to determine if it is above or below a normal level of the at least one biomarker, and administering to the subject a pharmaceutical composition comprising at least one steroid hormone in amount effective to restore the levels of the at least one biomarker to normal level. In one embodiment, the at least one steroid hormone comprises DHT. In an embodiment, the biomarker comprises hepatic protein or mRNA levels of CD36, VLDLR, Ces3, RGN, and LRP1, and hepatic TG and cholesterol levels. In one embodiment the liver disease or disorder comprises non-alcoholic fatty liver disease (NAFLD). In one embodiment, the subject at risk is a woman. In one embodiment, the subject at risk is a woman with NAFLD. In one embodiment, the subject at risk is a woman who displays at least one sign or symptom associated with PCOS. In one embodiment, the at least one sign or symptom associated with PCOS comprises hyperandrogenism.

In one embodiment, said method comprises oral administration of 45 mg of DHT per day to the subject. In one embodiment, said method comprises oral administration of 30 mg to 60 mg of DHT per day to the subject. In one embodiment, said method comprises oral administration of 20 mg to 75 mg of DHT per day to the subject. In one embodiment, said method comprises oral administration of about 60 mg to 180 mg of DHT per day to the subject. In one embodiment, said method comprises oral administration of about 40 mg to 200 mg of DHT per day to the subject. In one embodiment, said method comprises oral administration of about 50 mg, 75 mg, 100 mg, or 120 mg of DHT per day to the subject. In one embodiment, said method comprises any ophthalmic, oral, rectal, vaginal, parenteral, topical, pulmonary, intranasal, buccal, intravenous, intracerebroventricular, intradermal, intramuscular, or any other another suitable route of administration known in the field.

In one embodiment, said method comprises administration of DHT once per day to the subject for 1-3 weeks. In one embodiment, said method comprises administration of DHT once per day to the subject for 3-6 weeks. In one embodiment, said method comprises administration of DHT once per day to the subject for 6-9 weeks. In one embodiment, said method comprises administration of DHT once per day to the subject for 9-12 weeks.

In one embodiment, said method comprises administration of DHT 1-3 times per week to the subject for 1-3 weeks. In one embodiment, said method comprises administration of DHT 1-3 times per week to the subject for 3-6 weeks. In one embodiment, said method comprises administration of DHT 1-3 times per week to the subject for 6-9 weeks. In one embodiment, said method comprises administration of DHT 1-3 times per week to the subject for 9-12 weeks. In one embodiment, said method comprises administration of DHT 1-3 times per week to the subject for 12-15 weeks. In one embodiment, said method comprises administration of DHT 1-3 times per week to the subject for 15-18 weeks. In one embodiment, said method comprises administration of DHT 1-3 times per week to the subject for 18-21 weeks. In one embodiment, said method comprises administration of DHT 1-3 times per week to the subject for 21-24 weeks. In one embodiment, said method comprises administration of DHT 1-3 times per week to the subject for 24-27 weeks. In one embodiment, said method comprises administration of DHT 1-3 times per week to the subject for 27-30 weeks. In one embodiment, said method comprises administration of DHT 1-3 times per week to the subject for 30-33 weeks. In one embodiment, said method comprises administration of DHT 1-3 times per week to the subject for 33-36 weeks. In one embodiment, said method comprises administration of DHT 1-3 times per week to the subject for 36-39 weeks. In one embodiment, said method comprises administration of DHT 1-3 times per week to the subject for 39-42 weeks. In one embodiment, said method comprises administration of DHT 1-3 times per week to the subject for 42-45 weeks. In one embodiment, said method comprises oral administration of DHT 1-3 times per week to the subject for 45-48 weeks. In one embodiment, said method comprises administration of DHT 1-3 times per week to the subject for 48-51 weeks. In one embodiment, said method comprises administration of DHT 1-3 times per week to the subject for 51-54 weeks.

In one embodiment, treating the liver disease or disorder, or a symptom thereof, in a subject in need of treatment, comprises restoring the level of at least one biomarker of the liver disease or disorder to normal level, wherein the at least one biomarker comprises hepatic protein or mRNA levels of CD36, VLDLR, Ces3, RGN, and LRP1, and hepatic TG and cholesterol levels. Accordingly, in one embodiment, said method comprises administration of DHT until restoration of the at least one biomarker level to normal level is observed.

In one aspect, the at least one biomarker further comprises ALP, ALT, AST, GGT, LDL, liver biopsy, inflammation biomarkers, non-HDL cholesterol, hematocrit, hemoglobin, lipoprotein phospholipase A2, bilirubin, albumin, SHBG, imaging biomarkers, liver histology biomarkers, biomarkers in the literature related to the disease and condition described herein, and liver damage biomarkers.

In one embodiment, treating the liver disease or disorder, or a symptom thereof, in a subject in need of treatment, comprises restoring the level of at least one biomarker of the liver disease or disorder to normal level, wherein the at least one biomarker comprises any peptide, polypeptide, protein, nucleic acid, lipid, steroid, carbohydrate or any other biological molecule known to be dysregulated in liver disease or disorder, wherein the dysregulation comprises an alteration in level of the molecule.

In an embodiment, any appropriate biomarker can be used in the methods and compositions described herein (including e.g., imaging analysis, histology, genetic, microRNA). Specific biomarkers are described herein and in the Examples. Additional biomarkers, including some that are described herein elsewhere, are illustrated below.

In one embodiment, the methods and compositions described herein, are useful for treating a subject in need of treatment e.g., having NAFLD or at risk of having NAFLD or having fatty liver disease or having liver disease. Thus, the methods and compositions described herein can be used to treat NAFLD (or at risk of having NAFLD or having fatty liver disease or having liver disease) of any grade or type. In one aspect, the methods and compositions as disclosed herein, reduce liver fat or reduce the rate of increase in liver fat. In one aspect, the methods and compositions can reduce liver fat by greater than 5%, 10%, 15%, 20%, 25% or 30% in at least 1, 5, 10, 20, 30, 40, or 50% of subjects treated. In one aspect, the biomarker (liver fat) is determined as a % mean relative reduction in liver fat reduction with MRI-PDFF (Magnetic Resonance Imaging-Derived Proton Density Fat Fraction). In one aspect, the methods and compositions reduce liver triglyceride concentration or decrease the rate of increase liver triglyceride concentration. In one aspect, the biomarker (liver triglyceride concentration) is measured by NMRS (magnetic resonance spectroscopy). In one aspect, the method and composition can improve or ameliorate diffuse liver disease due to liver fat or iron deposition by 5%, 10%, 15%, 20% or 25% or more. In one aspect, non-invasive magnetic resonance imaging (MRI-PDFF) is sued for the evaluation of liver fat or iron deposition. In one aspect, the methods and compositions disclosed herein, ameliorates fatty infiltration (e.g., reduces fatty infiltration or reduces the rate of increase of fatty infiltration). In one aspect, abdominal ultrasound is used to determine fatty infiltration. In one aspect, CT (computed tomography) is used to assess the liver biomarker. In one aspect, the methods and compositions described herein improve liver stiffness (LS)/fibrosis. In one aspect, improved liver stiffness/fibrosis is at least a 5, 10, 15, 20 or 25% reduction in MRE (Magnetic Resonance Elastography)-stiffness. In one aspect, ultrasound based elastography is used for the assessment of liver tissue stiffness in fibrosis. In one aspect, transient elastography (TE) (e.g., Fibroscan®, Echosens, Paris) is used (ultrasound-based elastography) to determine liver stiffness. In one aspect, pSWE (Elastography point quantification, ElastPQ™, Phillips) or ARFI imaging (Virtual touch tissue Quantification™, Siemens) is (ultrasound-based elastography techniques) used to assess liver stiffness. In one aspect, liver stiffness is monitored via Real Time TE (RTE) which is a qualitative assessment of liver stiffness. Magnetic resonance elastography (MRE) can determine liver stiffness by analysis of mechanical waves propagating through the liver. In one aspect, the methods and compositions disclosed herein reduce liver stiffness by 5, 10, 20, 30, 40, 50, 60 or 75% or more. In one aspect, the methods and compositions disclosed herein reduce the rate of increase of liver stiffness by 5, 10, 20, 30, 40, 50, 60 or 75% or more. In one aspect, the method and compositions disclosed herein improve on liver fibrosis as measured by non-invasive LiverMultiscan. In one aspect, the method and compositions reduce hepatic steatosis as monitored by imaging studies along with decreasing in ALT, CK18, or both. In one aspect, the methods and compositions described herein improve or ameliorate liver fibrosis as assessed by histology and reduces risk of progression to cirrhosis. In one aspect, the methods and compositions described herein improve by at least 1 or 2 points (e.g., decrease) the NAFLD activity score (NAS). In one aspect, the methods and compositions described herein improve at least a point reduction in either lobular inflammation or hepatocellular ballooning. In one aspect, the methods and compositions improve or resolve NAFLD on overall histopathological interpretation by an experienced pathologist (e.g., target score of 0 on ballooning and 0 or 1 for inflammation, but no greater than 1) and no worsening of fibrosis (e.g., no one stage increase in on fibrosis score). In one aspect, the methods and compositions described herein reverse NAFLD (e.g., change from NAS score from 5 to 3) with no evidence of progression to advanced fibrosis (e.g., stage 3 or 4). In one aspect, the methods and compositions described herein improve fibrosis (e.g., lessen) without worsening of NAS or no progression of steatohepatitis (or reduce progression of steatohepatitis). In one aspect, the methods and compositions described herein do not increase NAFLD activity as assessed by NAS and lessen or reverse fibrosis. In one aspect, the methods and compositions described herein prevent the development of cirrhosis or lessen the likelihood of progression to cirrhosis. In one aspect, the methods and compositions described herein slowing histological progression to cirrhosis. In one aspect, the methods and compositions described herein improve event free survival (e.g., as assessed by all-cause mortality, new decompensation events, MELD score progression). In one aspect, the methods and compositions described herein decrease in all-cause mortality. In one aspect, the methods and compositions described herein decrease in liver specific mortality. In one aspect, the methods and compositions described herein decrease liver transplantation rates. In one aspect, the methods and compositions described herein increase 12-month survival rates. In one aspect, the methods and compositions described herein increase event free survival (EFS) at week 52 as Assessed by Time to the First Clinical Event. In one aspect, the methods and compositions described herein decrease events/rate of events of decompensation in compensated patients. In one aspect, the methods and compositions described herein decrease rate of ascites events or ascites grade. In one aspect, the methods and compositions described herein decrease rate of HE or progress in HE (e.g., as assessed by MM). In one aspect, the methods and compositions described herein decrease in hospital admissions or hospital admission rates. In one aspect, the methods and compositions described herein decrease unscheduled clinic or ER visit. In one aspect, the methods and compositions described herein decrease the number of tests performed. In one aspect, the methods and compositions described herein decrease lost work days. In one aspect, the methods and compositions described herein decrease infection rates or number of events. In one aspect, the methods and compositions described herein improve the Child-Pugh-Turcotte Score. In one aspect, the methods and compositions described herein lower Progression from A to B In one aspect, the methods and compositions described herein do not worsen CPT. In one aspect, the methods and compositions described herein improve CPT score by at least 1 or 2 points. In one aspect, the methods and compositions described herein improve MELD score. In one aspect, the methods and compositions described herein lessen responder progression to higher MELD score. In one aspect, the methods and compositions described herein do not worsen MELD score. In one aspect, the methods and compositions described herein improve MELD score by at least one or two points. In one aspect, the methods and compositions described herein lower % of subjects achieving transplantation qualifying MELD score of 14. In one aspect, the methods and compositions described herein increase in MELD score from <12 to 15 or higher. In one aspect, the methods and compositions described herein improve the hepatic venous pressure gradient (HVPG). In one aspect, the methods and compositions described herein reduce of proportion of subjects that progress to HVPG >10 mm. In one aspect, the methods and compositions described herein lower HVPG <10 mm. In one aspect, the methods and compositions described herein reduce HVPG by 5, 10, 15, 20, 25 or 30% or more. In one aspect, the methods and compositions described herein improve body composition. In one aspect, the methods and compositions described herein improve bone density (e.g., one or more of femoral, lumbar, neck, and total bone mass. In one aspect, the methods and compositions described herein improve anemia. In one aspect, the methods and compositions described herein improve gynecomastia (e.g., lessen).

In one embodiment, a method of reducing progression or the risk of progression of NAFLD to cirrhosis a subject having or suspected of having NAFLD is provided said method comprising oral administration of a pharmaceutical composition having a testosterone ester (e.g., testosterone undecanoate or testosterone tridecanoate) to said subject. In one aspect, the subject is in need of treatment e.g., has NAFLD or is at risk of having NAFLD progressing to cirrhosis. In one aspect, the subject in need of treatment has one or more biomarkers (or clinical characteristics) indicating an increased risk of NAFLD progressing to cirrhosis. In one aspect, the one or more biomarkers are outside the normal range. In one aspect, the one or more biomarkers are above the normal range but below 2 times the upper limit of the normal range. In one aspect of this method, the subject has PCOS.

In one aspect, the biomarker is one or more chosen from ALP, ALT, AST, GGT, triglycerides, LDL, cholesterol, liver biopsy, inflammation biomarkers, non-HDL cholesterol, hematocrit, hemoglobin, lipoprotein phospholipase A2, bilirubin, albumin, SHBG, any specific biomarkers disclosed in Example 1, imaging biomarkers, liver histology biomarkers, biomarkers in the literature related to the disease and condition described herein, and liver damage biomarkers. In one aspect of this embodiment, the subject has one or more of the following comorbidities: obesity, type 2 diabetes, dyslipidemia, cardiovascular disease, thyroid dysfunction, chronic kidney disease, liver disease, osteoporosis, hypogonadism, hypertension, sarcopenia, or cachexia.

The methods and compositions described herein are also directed to methods of inhibiting or reducing the likelihood of liver injury in a patient at risk for same occurring from or secondary to a variety of etiologies especially including hepatitis (all forms, especially including hepatitis viral), non-alcoholic fatty liver diseases (NAFLD), including non-alcoholic steatohepatitis (NASH), NAFLD or NASH including primary NASH, NASH secondary to liver transplantation (NASH post-liver transplantation), preservation injury of donated organs, acute and chronic liver transplant rejection and metabolic conditions including, for example, Wilson's disease, hemochromatosis, and alpha one antitrypsin deficiency represent alternative aspects of the present invention. In this method, an effective amount of a composition described herein is administered to a patient at risk for liver injury as described above in order to inhibit or reduce the likelihood of liver injury as described above. As a consequence of the actions of compounds according to the present invention in reducing and/or inhibiting liver injury, certain complications of liver injury may be reduced including, for example, liver failure, liver shock, obstructive jaundice, cirrhosis, including primary biliary cirrhosis, primary sclerosing cholangitis, portal hypertension, ascites, variceal bleeding, encephalopathy, depression, malaise, renal disease, arthritis, portal vein thrombosis, and budd chiari. The present disclosure is also directed to treating liver injury and/or reducing the likelihood of further liver injury associated with or occurring directly from or secondary to a variety of etiologies especially including hepatitis (all forms), cirrhosis (all types), non-alcoholic fatty liver diseases (NAFLD), including non-alcoholic steatohepatitis (NASH), NAFLD or NASH including primary NASH, NASH secondary to liver transplantation (NASH post-liver transplantation), preservation injury of donated organs, acute and chronic liver transplant rejection and metabolic conditions including, for example, Wilson's disease, hemochromatosis, and alpha one antitrypsin deficiency. In this method, an effective amount of an androgen receptor agonist according to the present invention is administered to a patient with liver injury and/or at risk for further liver injury as described above in order to treat, inhibit or reduce the likelihood of liver injury which occurs directly as a consequence of or secondary to one or more of the disease states and/or conditions as described above. As a consequence of the treatment methods described above, the occurrence and/or severity of one or more of the following conditions will be substantially reduced: liver failure, liver shock, obstructive jaundice, primary biliary cirrhosis, primary sclerosing cholangitis, portal hypertension, ascites, variceal bleeding, encephalopathy, depression, malaise, renal disease, arthritis, portal vein thrombosis and budd chiari.

### Compositions

In one embodiment, the present invention comprises compositions for treating a liver disease or disorder in a subject in need thereof, wherein the compositions comprise at least one steroid hormone. In one embodiment, the at least one steroid hormone comprises DHT. In one embodiment the liver disease or disorder comprises non-alcoholic fatty liver disease (NAFLD). In one embodiment, the subject is a woman. In one embodiment, the subject is a woman with NAFLD. In one embodiment, the subject is a woman who displays at least one sign or symptom associated with PCOS. In one embodiment, the at least one sign or symptom associated with PCOS comprises hyperandrogenism. In one embodiment, treating a liver disease or disorder in a subject in need thereof, comprises restoring the level of at least one biomarker of the liver disease or disorder to normal level, wherein the at least one biomarker comprises hepatic protein or mRNA levels of CD36, VLDLR, Ces3, RGN, and LRP1, and hepatic TG and cholesterol levels.

### Steroid Hormones

In one embodiment, the present invention comprises compositions and methods for treating a liver disease or disorder, or a symptom thereof, in a subject in need of treatment, the method comprising administering to the subject at least one steroid hormone in an amount effective for treating the liver disease or disorder. In one embodiment, the subject at risk is a woman. In one embodiment, the subject at risk is a woman with NAFLD. In one embodiment, the subject at risk is a woman who displays at least one sign or symptom associated with PCOS. In one embodiment, the at least one sign or symptom associated with PCOS comprises hyperandrogenism.

In one embodiment, the composition comprises at least one steroid hormone, wherein the at least one steroid hormone comprises dihydrotestosterone (DHT). In one embodiment, the at least one steroid hormone is any analog of DHT. In one embodiment, the at least one steroid hormone is any “DHT compound”, wherein “DHT compound” is intended to include DHT and analogues of DHT which have been chemically modified to retard the rate of catabolism or to enhance the androgenic potency of DHT. These chemical modifications of DHT include esterification of the 17β-hydroxyl group with any of several carboxylic acids to decrease the polarity of the molecule, making it more soluble in lipid vehicles used for injection and slowed release into the bloodstream. The longer the carbon chain in the ester, the more lipid soluble the steroid becomes and the more prolonged is the release of the steroid in vivo. Other dihydrotestosterone esters include those derived from aliphatic carboxylic acids, derivatives and analogues of dihydrotestosterone, short chain esters of DHT (e.g., proprionate and cypionate) as well as long and branched chain esters, and esters of DHT (e.g., 3-alkyloxy form of DHT). Other modifications of DHT which are encompassed by the invention also include alkylation at the 17α position. Such alkylated derivatives are are slowly catabolized by the liver. The alkyl group is not removed metabolically. Therefore, the alkylated derivative mediates the action of the hormone within cells. Preferred compounds of the invention include 5α-dihydrotestosterone (5α DHT), 5β-dihydrotestosterone (5β DHT), and esters, analogues and/or derivatives thereof, such as 5α and 5β reduced analogues, biocompatible derivatives, such as alkanoic acid derivatives, 17 alkylated (e.g., methylated) derivatives of 5α and 5β DHT, and 5α and 5β DHT esters that are hydrolyzable in vivo. Examples of aliphatic carboxylic acids, from which the foregoing esters are derived, preferably include pelargonic acid, capric acid, undecanoic acid, lauric acid, tridecanoic acid, myristic acid, pentadecanoic acid, undecenoic acid, palmitic acid and the branched chain and cyclic analogues of these acids. The manufacture of these esters is set forth in U.S. Pat. Nos. 4,098,802 and 4,220,599 of van der Vies, which are both herein incorporated by reference.

In other aspects, the compositions of the disclosure comprise a dihydrotestosterone derivative. Dihydrotestosterone derivatives are known in the art and include, for example, steroidal compounds including the following A-B—C-D core structure:

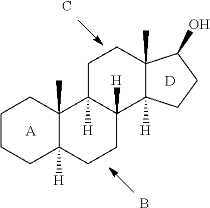

wherein the A-B—C-D core structure is substituted at any position with a substituent moiety, for example, a C1-6 straight or branched alkyl moiety, an C6-10 aryl moiety, or a 5- or 6-membered heteroaryl moiety that includes 1 or 2 heteroatoms selected from nitrogen, oxygen, and sulfur.

Dihydrotestosterone derivatives include, for example, mesterolone and drostanolone. In one embodiment, preferred dihydrotestosterone derivative is stanozolol.

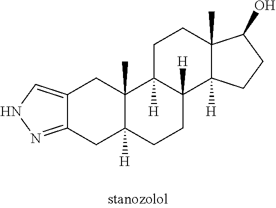

In some embodiments, the at least one steroid hormone comprises testosterone, 4-hydroxytestosterone, 11-ketotestosterone, boldenone, clostebol, 4-androstenediol, 4-dehydroepiandrosterone (4-DHEA), 5-androstenedione, 5-dehydroandrosterone (5-DHA), 11β-hydroxyandrostenedione (11β-OHA4), adrenosterone (11-ketoandrostenedione, 11-ka4), androstenediol (5-androstenediol, A5), androstenedione (4-androstenedione, A4), atamestane, boldione (1,4-androstadienedione), dehydroepiandrosterone (DHEA, 5-DHEA; prasterone, androstenolone), exemestane, formestane, plomestane, cloxotestosterone, quinbolone, silandrone, dihydrotestosterone (DHT); androstanolone, stanolone), 1-testosterone (dihydro-1-testosterone, dihydroboldenone), 11-ketodihydrotestosterone (11-KDHT), drostanolone, epitiostanol (epithioandrostanol), mesterolone, metenolone (methenolone, methylandrostenolone), nisterime, stenbolone, 1-androsterone (1-andro, 1-DHEA), 1-androstenediol (dihydro-1-androstenediol), 1-androstenedione (dihydro-1-androstenedione), 5α-androst-2-en-17-one, androsterone, epiandrosterone, mepitiostane, mesabolone, prostanozol, bolazine (di(drostanolone) azine), nandrolone (nortestosterone), 11β-methyl-19-nortestosterone (11β-MNT), dienolone, dimethandrolone, norclostebol, oxabolone, trenbolone (trienolone), trestolone (MENT), 7α-methyl-19-nor-4-androstenedione (ment dione, trestione), 19-nor-5-androstenediol, 19-nor-5-androstenedione, 19-nordehydroepiandrosterone, bolandiol (nor-4-androstenediol), bolandione (nor-4-androstenedione), dienedione (nor-4,9-androstadienedione), methoxydienone (methoxygonadiene), trendione (nor-4,9,11-androstatrienedione), bolmantalate (nandrolone adamantoate), bolasterone, calusterone, chlorodehydromethyltestosterone (CDMT), enestebol, ethyltestosterone, fluoxymesterone, formebolone, hydroxystenozole, metandienone (methandienone, methandrostenolone), methylclostebol (chloromethyltestosterone), methyltestosterone, oxymesterone, tiomesterone (thiomesterone), chlorodehydromethylandrostenediol (CDMA), chloromethylandrostenediol (CMA), methandriol (methylandrostenediol), methyltestosterone 3-hexyl ether, penmesterol (penmestrol), androisoxazole, desoxymethyltestosterone, furazabol, mestanolone (methyl-DHT), methasterone (methyldrostanolone), methyl-1-testosterone (methyldihydro-1-testosterone), methyldiazinol, methylepitiostanol, methylstenbolone, oxandrolone, oxymetholone, stanozolol, mebolazine (dimethazine, di(methasterone) azine), dimethyltrienolone (7α,17α-dimethyltrenbolone), dimethyldienolone (7α,17α-dimethyldienolone), ethyldienolone, ethylestrenol (ethylnandrol), methyldienolone, methylhydroxynandrolone (MOHN, MHN), metribolone (methyltrienolone, r-1881) mibolerone, norboletone, norethandrolone (ethylnandrolone, ethylestrenolone), norme thandrone (methylestrenolone, normethisterone), RU-2309 (18-methymetribolone, 17α-methyl-THG), tetrahydrogestrinone (THG), bolenol (ethylnorandrostenol), propetandrol, vinyltestosterone, norvinisterone (vinylnortestosterone), ethisterone (ethinyltestosterone), danazol (2,3-isoxazolethisterone), norethisterone (norethindrone), etynodiol (ethynodiol, 3β-hydroxynorethisterone), gestrinone (ethylnorgestrienone, r-2323), levonorgestrel ((−)-norgestrel), lynestrenol (3-deketonorethisterone), norgestrel (18-methylnorethisterone) norgestrienone (ethynyltrenbolone), tibolone (7α-methylnoretynodrel), quingestanol etynodiol diacetate (ethynodiol diacetate), norethisterone acetate (norethindrone acetate), norethisterone enanthate (norethindrone enanthate), quingestanol acetate, or any combination thereof.

The compositions of the present invention may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

The formulations of the pharmaceutical compositions may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to subjects of all sorts. Modification of pharmaceutical compositions suitable for administration to humans in order to render the compositions suitable for administration to various subjects is well understood, and the ordinarily skilled veterinary pharmacologist can design and perform such modification with merely ordinary, if any, experimentation. Individuals to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intraocular, intramuscular, intradermal and intravenous routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. A unit dose is discrete amount of the pharmaceutical composition comprising a predetermined amount of the active ingredient. The amount of the active ingredient is generally equal to the dosage of the active ingredient which would be administered to an individual or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the individual treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Parenteral administration of a pharmaceutical composition includes any route of administration characterized by physical breaching of a tissue of an individual and administration of the pharmaceutical composition through the breach in the tissue. Parental administration can be local, regional or systemic. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and in some embodiments from about 1 to about 6 nanometers. Such compositions are conveniently in the form of dry powders for administration using a device comprising a dry powder reservoir to which a stream of propellant may be directed to disperse the powder or using a self propelling solvent/powder dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some embodiments having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. In some embodiments, the droplets provided by this route of administration have an average diameter in the range from about 0.1 to about 200 nanometers.

The formulations are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more additional ingredients.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more additional ingredients. Alternately, formulations suitable for buccal administration may comprise a powder or an aerosolized or atomized solution or suspension comprising the active ingredient. In some embodiments, such powdered, aerosolized, or aerosolized formulations, when dispersed, have an average particle or droplet size in the range from about 0.1 to about 200 nanometers, and may further comprise one or more additional ingredients.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Dosage and Formulations (Compositions)

Administration of the composition in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent or modified cell may be directly injected into the tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The agents of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the compositions of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the compositions in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Kits

The invention also includes a kit comprising the compositions described elsewhere herein, or combinations thereof, of the invention and an instructional material which describes, for instance, administering the composition, or combinations thereof, to an individual as a therapeutic treatment as described elsewhere herein. In an embodiment, this kit further comprises a (preferably sterile) pharmaceutically acceptable carrier suitable for dissolving or suspending the therapeutic composition, comprising a steroid hormone, or combinations thereof, of the invention, for instance, prior to administering the composition to an individual. Optionally, the kit comprises an applicator for administering the composition.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Dihydrotestosterone Prevented Nonalcoholic Fatty Liver Disease Induced with Western Diet in Obese Female Mice

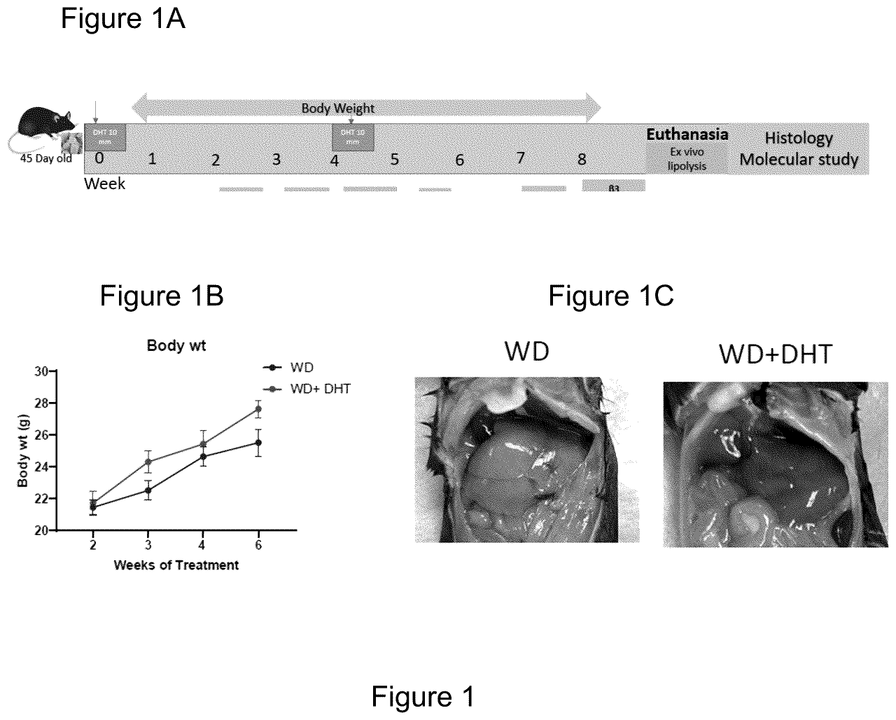

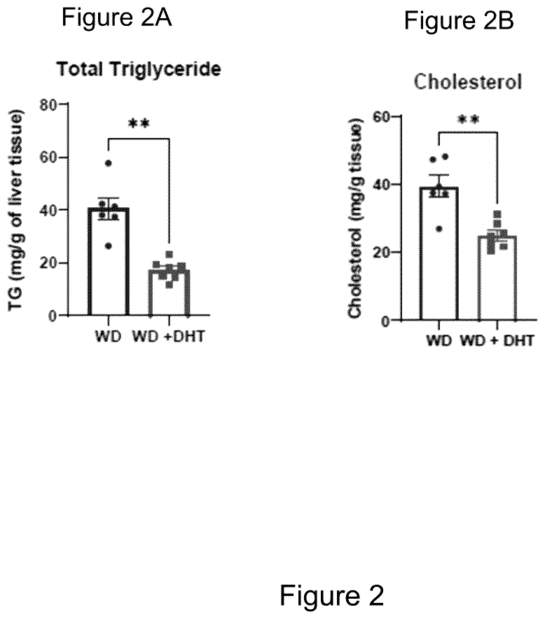

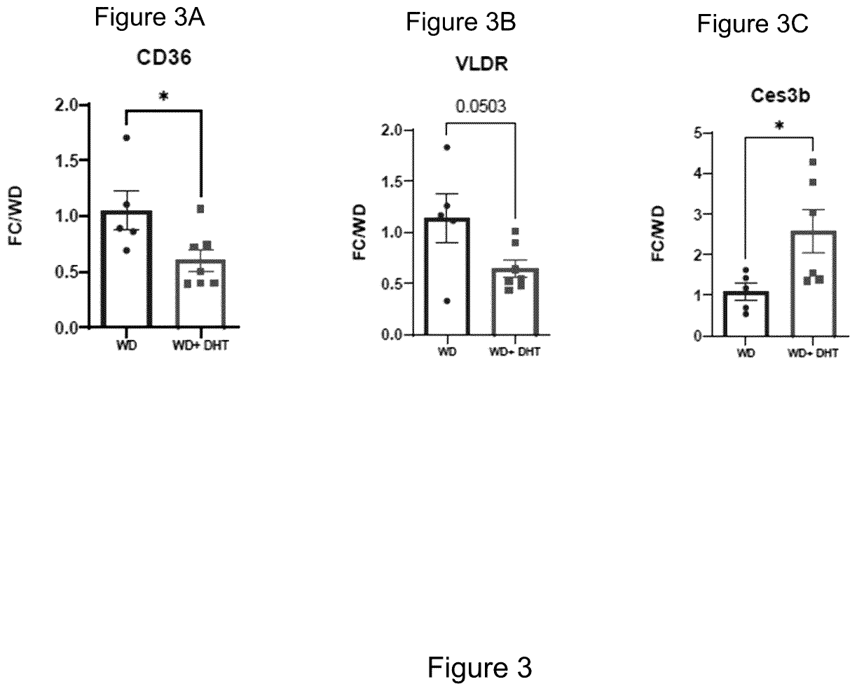

Significantly elevated levels of triglyceride (TG) in circulation and lower levels of TG and cholesterol in the liver of mice treated with dihydrotestosterone (DHT) with western diet (WD+DHT) compared to western diet alone group (WD) were observed (FIG. 1 through FIG. 4). Genes related to fatty acid transport into the liver and TG secretion from the liver such as CD36 and regucalcin were significantly altered in the livers of mice receiving DHT with western diet (FIG. 3). These studies show that in the mice receiving DHT in the presence of western diet, the liver however adopts a protective mechanism with dramatic decrease in lipid storage.

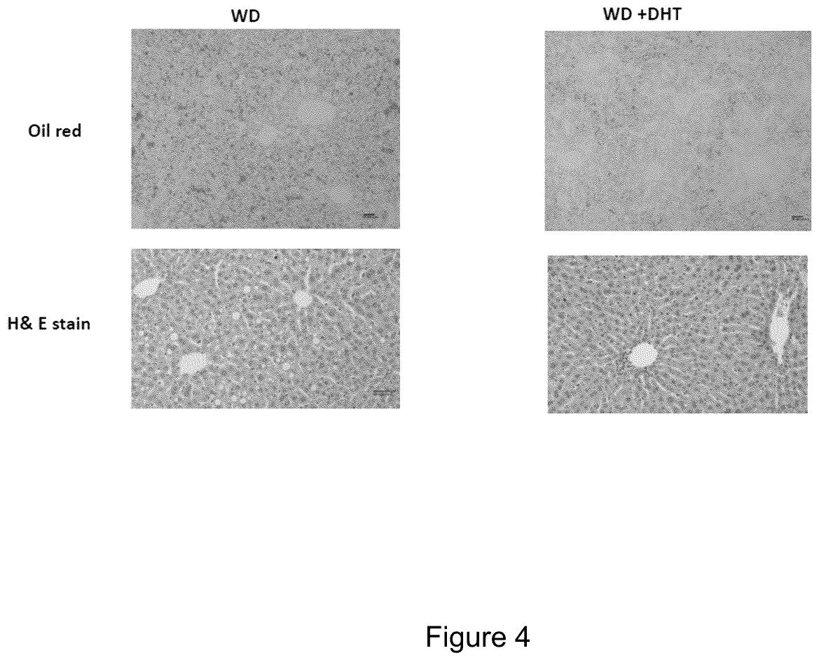

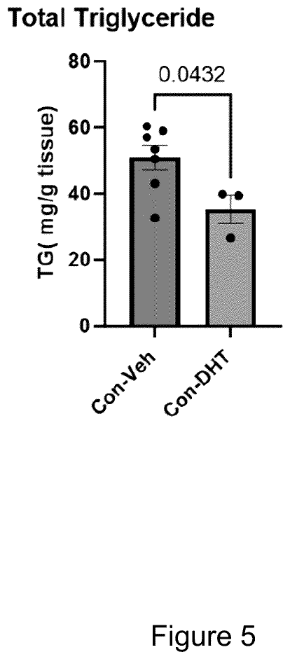

Adult female mice treated with DHT and with Western diet (WD) showed more impaired glucose intolerance at 3 weeks, along with more impaired pyruvate tolerance at 6-7 weeks compared to female mice with WD alone (FIG. 1 through FIG. 4). q-PCR or proteomics studies showed that genes related to fatty acid transport into the liver (CD36), triglyceride (TG) secretion from the liver (Ces31b), VLDR, regucalcin, and prolow-density lipoprotein-related protein 1 were significantly altered the livers of mice receiving DHT with WD compared to WD alone (FIG. 3). Histology studies showed that there is decreased lipid deposition in the livers of the DHT mice, while the brown adipose cell size was enlarged with more lipid droplet inside in the DHT with WD mice (FIG. 1C and FIG. 4). These studies show that in the mice receiving DHT in the presence of WD, under more free fatty acid release from adipose tissue, the liver however adopts a protective mechanism with decreased lipid storage (FIG. 1C and FIG. 4). Contrary to commonly held perspectives in the field, these surprisingly and unexpectedly demonstrate that DHT protected mice from fatty liver and/or corrected fatty liver defects/disease in mice (FIG. 1 through FIG. 4). Additionally, it was determined that liver shows lower triglyceride after mice are fed with WD for 8 weeks and then treated with DHT for 3 weeks under WD condition (11 weeks total WD), compared to liver of WD alone for 11 weeks (FIG. 5).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating a liver disease or disorder, or a symptom thereof, in a subject in need of treatment, the method comprising administering to the subject at least one steroid hormone in an amount effective for treating the liver disease or disorder.

2. The method of claim 1, wherein the subject is a woman, and wherein the liver disease or disorder is non-alcoholic fatty liver disease (NAFLD).

3. The method of claim 1, wherein the subject is a woman, and wherein the woman further displays at least one clinical feature associated with polycystic ovary syndrome (PCOS).

4. The method of claim 3, wherein the at least one clinical feature associated with PCOS comprises irregular or no menstrual periods, heavy periods, excess body and facial hair, acne, pelvic pain, difficulty getting pregnant, and patches of thick, darker, velvety skin, ovarian cysts, enlarged ovaries, excess androgen, weight gain, hirsutism, type 2 diabetes, obesity, obstructive sleep apnea, heart disease, mood disorders, endometrial cancer, hyperandrogenism, or any combination thereof.

5. The method of claim 1, wherein the liver disease or disorder comprises non-alcoholic fatty liver disease (NAFLD), nonalcoholic steatohepatitis (NASH), liver fibrosis, alcoholic liver disease, hepatitis, NASH with cirrhosis, and comorbidities of testosterone abnormalities.

6. The method of claim 1, wherein the liver disease or disorder comprises NAFLD.

7. The method of claim 1, wherein the at least one steroid hormone comprises dihydrotestosterone (DHT).

8. The method of claim 1, wherein the method restores the level of at least one biomarker of the liver disease or disorder in the subject.

9. The method of claim 9, wherein the at least one biomarker comprises hepatic protein or mRNA levels of Cluster of Differentiation 36 (CD36), Very Low Density Lipoprotein Receptor (VLDLR), Carboxylesterase 3 (Ces3), Regucalcin (RGN), Prolow-density lipoprotein receptor-related protein 1 (LRP1), and hepatic levels triglyceride (TG) and cholesterol levels.

10. A composition for treating a liver disease or disorder in a subject in need thereof, wherein the composition comprises at least one steroid hormone.

11. The composition of claim 10, wherein the subject is a woman, and wherein the liver disease or disorder is non-alcoholic fatty liver disease (NAFLD).

12. The composition of claim 10, wherein the subject is a woman, and wherein the woman further displays at least one clinical feature associated with PCOS.

13. The composition of claim 12, wherein the at least one clinical feature associated with PCOS comprises irregular or no menstrual periods, heavy periods, excess body and facial hair, acne, pelvic pain, difficulty getting pregnant, and patches of thick, darker, velvety skin, ovarian cysts, enlarged ovaries, excess androgen, weight gain, hirsutism, type 2 diabetes, obesity, obstructive sleep apnea, heart disease, mood disorders, endometrial cancer, hyperandrogenism, or any combination thereof.

14. The composition of claim 10, wherein the liver disease or disorder comprises NAFLD, NASH, liver fibrosis, alcoholic liver disease, hepatitis, NASH with cirrhosis, and comorbidities of testosterone abnormalities.

15. The composition of claim 10, wherein the liver disease or disorder comprises NAFLD.

16. The composition of claim 10, wherein the at least one steroid hormone comprises DHT.

17. The composition of claim 10, wherein the composition restores the level of at least one biomarker of the liver disease or disorder in the subject following administration to the subject.

18. The composition of claim 17, wherein the at least one biomarker comprises hepatic protein or mRNA levels of CD36, VLDLR, Ces3, RGN, and LRP1, and hepatic TG and cholesterol levels.

19. The composition of claim 10, wherein the composition is a pharmaceutical composition.

20. The composition of claim 19, further comprising at least one pharmacologically acceptable excipient.

21. A method of preventing a subject at risk of developing a liver disease or disorder from developing the liver disease or disorder, the method comprising:
a) identifying a subject at risk, wherein the identifying comprises:
i) measuring the level of at least one biomarker associated with the liver disease or disorder in a subject;
ii) comparing the level of the at least one biomarker to determine to normal levels of the at least one biomarker; and
iii) determining that the subject is at risk if the levels of the at least one biomarker is above or below the normal levels;
and
b) administering to the subject at risk at least one steroid hormone in an amount effective for treating the liver disease or disorder.

22. The method of claim 21, wherein the subject is a woman, and wherein the liver disease or disorder is non-alcoholic fatty liver disease (NAFLD).

23. The method of claim 21, wherein the subject is a woman, and wherein the woman further displays at least one clinical feature associated with PCOS.

24. The method of claim 23, wherein the at least one clinical feature associated with PCOS comprises irregular or no menstrual periods, heavy periods, excess body and facial hair, acne, pelvic pain, difficulty getting pregnant, and patches of thick, darker, velvety skin, ovarian cysts, enlarged ovaries, excess androgen, weight gain, hirsutism, type 2 diabetes, obesity, obstructive sleep apnea, heart disease, mood disorders, endometrial cancer, hyperandrogenism, or any combination thereof.

25. The method of claim 21, wherein the liver disease or disorder comprises NAFLD.

26. The method of claim 21, wherein the at least one steroid hormone comprises DHT.

27. The method of claim 21, wherein the method of preventing a liver disease or disorder comprises restoring levels of the at least one biomarker in the subject at risk.

28. The method of claim 21, wherein the at least one biomarker comprises hepatic protein or mRNA levels of CD36, VLDLR, Ces3, RGN, and LRP1, and hepatic TG and cholesterol levels.
